(19) United States
(12) Patent Application Publication
(10) Pub. No.: US 2011/0251397 A1
(43) Pub. Date: Oct. 13, 2011

(54) METHOD FOR PREPARATION OF 3-METHYLPYRIDINE

(76) Inventors: Anna Maria Cornelia Francisca Castelijns, Spaubeek (NL); Pierre Louis Woestenborghs, Dilsen (BE); Henricus Wilhelmus Leonardus Marie Vaessen, Wijnandsrade (NL)

(21) Appl. No.: 13/119,059

(22) PCT Filed: Sep. 8, 2009
(86) PCT No.: PCT/EP09/61625

§ 371 Date: Jun. 8, 2011

(30) Foreign Application Priority Data
Sep. 26, 2008 (EP) 08105445.4

(51) Int. Cl. C07D 213/16

(52) U.S. Cl. 546/252; 546/251

(57) ABSTRACT

The present invention relates to a method for the preparation of 3-methylpyridin by reacting 2-methyl-2,5-di-aminopentane and/or 3-methylpiperidin at about atmospheric pressure and a temperature of 180° C. to 400° C. in a hydrogen atmosphere free of oxygen gas in the presence of a suitable catalyst and of water and/or a volatile alcohol.

The present invention relates to a method for the preparation of 3-methylpyridin.

Such a method is described in German Offenlegungsschrift 2519529. According to this document, 3-methylpyridine (compound of formula [1]) can be prepared by reacting hexane-1,5-diamine (compound of formula [2]) or a mixture of compound of formula [2] and 3-methylpiperidine (compound of formula [3]) at about atmospheric pressure and a temperature of 200° C. to 400° C. in a hydrogen atmosphere free of oxygen gas in the presence of a palladium catalyst.

This reaction may result in a good yield. However, it was found that when the reaction is being carried out in a continuous mode during many days the yield of 3-methylpyridin dramatically decreases over time.

According to the present invention such a decrease of yield of 3-methylpyridin can be dramatically diminished.

Accordingly, the present invention relates to a process for the preparation of a compound of the formula [1]

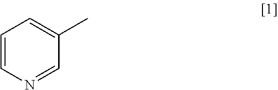

by reacting a compound of formula [2]

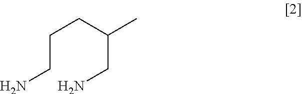

and/or a compound of formula [3]

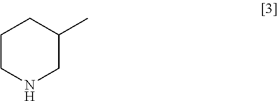

at about atmospheric pressure and at a temperature of 180° C. to 400° C. in an hydrogen atmosphere free of oxygen gas in the presence of a suitable catalyst which process is characterized in that the reaction mixture contains water and/or an alcohol.

The ratio of the compounds according to formula [2] and [3] in the reaction mixture may vary widely. Hence, the mixture may contain between 0 and 0.100 mol % of the compound of formula [2] and between 100 and 0 mol % of the compound of formula [3].

The alcohol can be any alcohol volatile under the reaction conditions applied. Preferably it is a C1to C5 alcohol. Most preferably methanol is used.

Preferably, the water and/or alcohol is present in the reaction mixture in a molar ratio relative to the total of the compounds [2] and/or [3] of between 1:1.5 and 1:180.

In the context of the present invention with “about atmospheric pressure” is meant a pressure between about 40 and 500 kPa. A compound of formula [2] and/or a compound of formula [3] suitably can be prepared by hydrogenation of a compound of formula [4]

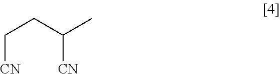

as described in German Offenlegungsschrift 2514004, the content of which is incorporated herein by reference. The molar ratio of the compounds of the formula [2] and [3] in the resulting mixture is not critical for further use in the reaction according to the present invention.

A “suitable catalyst” for the process of the present invention is a metal catalyst from Group 8 or 1B metals which is able to catalyze a heterogeneous hydrogenation reaction.

Preferably, in the method of the present invention use is made of a palladium catalyst. Generally, the palladium is used in the process of the present invention dispersed or adsorbed on the surface of an inert support. Such supports are well known and include for example active carbon, silica, alumina, diatomaceous earth, kieselguhr and mixtures thereof. Palladium constitutes generally between 0.2 and 12% w/w of such carrier-bound catalysts. The amount of palladium catalyst relative to the amount of reactants is not critical as long as it is sufficient to establish complete reduction of the reactants, and can readily be found by the man skilled in the art on the basis of simple experiments.

Following the reaction according to the present invention the desired compound of formula 1 can be further purified and optionally freed from contaminants such as unreacted reactants of formula [2] and/or [3] and/or possible side products by methods known in the art.

The compound of formula [1] is useful as an intermediate in the production of pharmaceutically active and crop protection compounds.

### EXAMPLE

### Comparative Example of Preparation of 3-Methylpyridine with and Without Water or Alcohol

A gaseous mixture of hexane-1,5-diamine (Dytek A) and H2 was passed over a Pd/Al2O3 catalyst at 0.15 ml/g. cat/hr (LHSV (Liquid Hourly Space Velocity)=0.15). The H2 feed was 13 NI/hr. The temperature of the catalyst bed was 280° C.

After 36 hrs of reaction the conversion of hexane-1,5-diamine was 100% and the selectivities to 3-methylpyridine and 3-methylpiperidine were resp. 97.3% and 1.3%. Small amounts of 2-amino-3-methylpyridine and 2-amino-5-methylpyridine were also detected.

After a total reaction time of 38 hrs. the temperature of the catalyst bed was increased to 300° C. The results are summarized in Table 1.

TABLE 1

| Total | | | Selectivity | Selectivity | Sum |
|---|---|---|---|---|---|
| reaction | T cat. | Conv. | to 3-Me- | to 3-Me- | selectivity |
| time (hr) | (° C.) | (%) | pyridine (%) | piper-idine (%) | (%) |
| 54.1 | 300 | 100 | 92.8 | 0.2 | 93 |
| 60.5 | 300 | 100 | 88.6 | 1.5 | 90.1 |
| 77.3 | 300 | 100 | 59.2 | 15.7 | 74.9 |
| 79.6 | 300 | 100 | 53.6 | 22 | 75.6 |

Then the temperature of the catalyst bed was decreased to 270° C. which resulted, after a total reaction time of 82.1 hrs in a conversion of hexane-1,5-diamine of 100% and selectivities to 3-methylpyridine and 3-methylpiperidine of resp. 49.4% and 49.7%. From these figures it is obvious that the catalyst is rapidly deactivated under the reaction conditions applied.

Then, 6.7 wt. % of water was added to the hexane-1,5-diamine and this mixture was dosed in the with a LHSV of about 0.15 to the catalyst. The results are summarized in Table 2.

TABLE 2

| Total | | | Selectivity | Selectivity | Sum |
|---|---|---|---|---|---|
| reaction | T cat. | Conv. | to 3-Me- | to 3-Me- | selectivity |
| time (hr) | (° C.) | (%) | pyridine (%) | piper-idine (%) | (%) |
| 98.6 | 270 | 100 | 89.4 | 1 | 90.4 |
| 113 | 270 | 100 | 93.8 | 2.1 | 95.9 |
| 136.9 | 270 | 100 | 94.2 | 2.7 | 96.9 |
| 193.8 | 270 | 100 | 94.4 | 3.5 | 97.9 |
| 324.3 | 270 | 100 | 94.3 | 2.1 | 96.5 |

Evidently, the catalyst has regained its activity by the presence of water.

After a total reaction time of 330 hrs., the water in the feed was replaced by 5 wt. % of methanol. The results obtained in this experiment are summarized in Table 3.

TABLE 3

| Total | | | Selectivity | Selectivity | Sum |
|---|---|---|---|---|---|
| reaction | T cat. | Conv. | to 3-Me- | to 3-Me- | selectivity |
| time (hr) | (° C.) | (%) | pyridine (%) | piper-idine (%) | (%) |
| 349.8 | 270 | 100 | 90.9 | 6.6 | 97.4 |
| 371.5 | 270 | 100 | 91.4 | 6.2 | 97.6 |

It can be concluded that the results with an alcohol are highly comparable with the results attained in the presence of water.

Then, the LHSV was decreased to 0.05 ml/g cat/hr. The results are summarized in Table 4.

TABLE 4

| Total | | | Selectivity | Selectivity | Sum |
|---|---|---|---|---|---|
| reaction | T cat. | Conv. | to 3-Me- | to 3-Me- | selectivity |
| time (hr) | (° C.) | (%) | pyridine (%) | piper-idine (%) | (%) |
| 486.5 | 270 | 100 | 92.6 | 0.7 | 93.3 |
| 530.4 | 270 | 100 | 95.8 | 0.6 | 96.4 |
| 605.8 | 270 | 100 | 94.3 | 0.4 | 94.7 |

1. Method for the preparation of a compound of the formula [1] 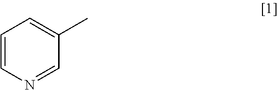
by reacting a compound of formula [2] 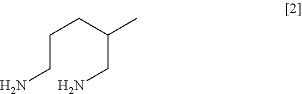
and/or a compound of formula [3] 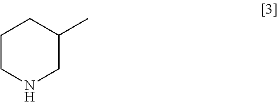
at about atmospheric pressure and a temperature of 180° C. to 400° C. in an hydrogen atmosphere free of oxygen gas in the presence of a suitable catalyst, characterized in that the reaction mixture contains water and/or an alcohol.

2. Method according to claim 1, characterized in that the alcohol is a C1 to C5 alcohol.

3. Method according to claim 1 characterized in that the water and/or alcohol is present in the reaction mixture in a molar ratio relative to the compound [2] and or [3] of between 1:1.5 and 1:180

4. Method according to claim 1 characterized in that the pressure is between 40 and 500 kPa.

5. Method according to claim 1 characterized in that the catalyst is selected from the Group 8 and 1B metals.

6. Method according to claim 1 characterized in that the catalyst is a palladium catalyst.
